(19) United States
(12) Patent Application Publication
(10) Pub. No.: US 2020/0089911 A1
(43) Pub. Date: Mar. 19, 2020

(54) INFORMATION PROCESSING SYSTEM

(71) Applicant: Keysoft, Inc., Kanagawa (JP)

(72) Inventor: Yoshimitsu Kagiwada, Kanagawa (JP)

(21) Appl. No.: 16/609,171

(22) PCT Filed: Mar. 30, 2018
(86) PCT No.: PCT/JP2018/013972

§ 371 Date: Oct. 28, 2019

(30) Foreign Application Priority Data
Apr. 28, 2017 (JP) 2017090532

(51) Int. Cl. G06F 21/62; G06F 16/9035; G06F 21/60; G16H 10/60; G06F 21/31

(52) U.S. Cl. CPC G06F 21/6245; G06F 16/9035; G06F 21/31; G16H 10/60; G06F 21/604

(57) ABSTRACT

An information processing system is provided that publicizes individual-related information so that the individual cannot be identified. The information processing system includes: a database for storing individual-related publicized information; an information publicizing server; and a person meta data generator. In the information processing system connected to the outside via a network, a database is configured to store each piece of individual related publicized information together with a flag showing whether or not the information is publicized so that the individual cannot be identified. The person meta data generator is configured to generate person meta data based on the publicized information to be publicized so that the individual cannot be identified. The information publicizing server is configured to separately publicize, among the publicized information stored in the database, the publicized information that is not shown to be publicized so that the individual cannot be identified and the generated person meta data.

### TECHNICAL FIELD

The present invention relates to an information processing system. The invention relates to an information processing system to process individual-related information.

### BACKGROUND ART

In recent years, attention has been paid on the usefulness of the use of big data. The big data is various types and forms of unstructured data and atypical data for example and is a collection of data increasing in an accumulated manner day by day. Thus, data groups, which have been overlooked by the conventional technique because of the impossibility of managing such enormous data, are increasingly effectively used for a business purpose for example by being recorded and stored to be subjected to a quick analysis.

The big data is generated from SNSs (social networking services) such as FACEBOOK® or TWITTER® that generate big data including a great amount of information day by day and GPS information from smart phones for example.

The use of the big data can provide, for example, an appropriate understanding of the market needs for a product development purpose for example. Thus, the above-described data conventionally publicized on the Internet via SNSs for example is used and analyzed as big data.

It has been expected to use, as big data, information such as diagnosis records and prescriptions from medical examinees and patients in medical institutions such as hospitals. The use of the information including the diagnosis records and prescriptions as big data allows pharmaceutical companies to develop products more suitable for the market needs.

### SUMMARY OF INVENTION

However, information in SNSs is publicized on the Internet so that the information can be accessed by specific persons forming a community or can be accessed by unspecified persons. The information publicized so that the information can be accessed only by specific persons is hard to be used as big data because this information provides a limited access from the outside. The information publicized so that the information can be accessed by unspecified persons can be used as big data.

FIG. 1 is a schematic view illustrating the configuration of an SNS information processing system. FIG. 1 shows a network 190 such as the Internet connected to an SNS information processing system 100 and user terminals 160 and 162 as well as a search server 180 connected to the SNS information processing system 100 via the network 190.

The SNS information processing system 100 can be configured by one or more computers including: a processor such as a CPU; a semiconductor memory or a magnetic or optical memory; a wired or wireless communication device; an input apparatus such as a keyboard, an input pad, a mouse pointer, or a microphone; and an output apparatus such as a display, a printer, or a speaker.

The SNS information processing system 100 includes a database (DB) 102, an information publicizing server 104, a communication server 106, and an authentication server 108.

The database 102 is a database that stores information for the respective users of the SNS information processing system 100. The database 102 is retained in the memory and information is written/read therein in response to a request.

FIG. 2 illustrates an example of user information stored in the database 102. The database 102 includes the authentication information 202 (user ID, password), the registration information 204 (name/title, date of birth, address, telephone number, mail address, sex), and the publicized information 206 (e.g., profiles 1 and 2, blog article). The database 102 can include a contact list 208 (e.g., names/titles, mail addresses, and telephone numbers of other users). The example shown in FIG. 2 shows that the stored information is input/set by a user AAA via the user terminal 160.

The information publicizing server 104 is implemented by allowing a processor to execute a program such as an HTML (HyperText Markup Language) server. The information publicizing server 104 can publicize the user information stored in the database 102 at a predetermined address.

FIG. 3 illustrates an example of the user information publicized by the information publicizing server 104. FIG. 3 illustrates a status in which publicized information 206 of the user AAA stored in the database 102 shown in FIG. 2 is arranged and publicized in a blog format. By specifying an address aaa on the Internet, the blog of the user AAA can be accessed by a user terminal 162 of another user (e.g., a user BBB included in the contact list of the user AAA). The blog of the user AAA also can be searched using the search server 180. The example of the blog of FIG. 3 displays a region (button) that can be activated in order to send a message such as a mail to AAA and a region (button) to add a comment regarding a blog article. For example, the user BBB can access the publicized blog of the user AAA via the user terminal 162 to activate a message button by which a user interface is displayed on the user terminal 162 used to transmit a message to the mail address of the AAA (ID1@xxx) to provide the preparation and transmission of the message. The user BBB can activate a comment button to present a user interface UI for comment preparation on the user terminal 162 in which a comment regarding the publicized information can be written.

The communication server 106 is implemented by a processor for executing a program to support the Internet protocol (IP) communication and a communication device for example. The communication server 106 also can be a mail server program using a protocol such as a POP (Post Office Protocol)/SMTP (Simple Mail Transfer Protocol) or an IMAP (Internet Message Access Protocol) or a server executing a short message service SMS program. The communication server 106 also can be a file server to execute a protocol such as an FTP (File Transfer Protocol). The communication server 106 can communicate with the user terminals 160 and 162, the search server 180, the information publicizing server 104, an authentication server 108, or other mail servers or SNS servers or other file servers.

The authentication server 108 provides a function to authenticate a user of the information publicizing server 104 (by password collation for example) and a function to control the access to information stored in the database 102.

However, as shown in FIG. 3, some users do not want to publicize information in SNSs to include physical feature-related information such as the one regarding their physical conditions or health or medical histories. Specifically, some users do not want to publicize the physical feature-related information such as the one regarding their physical conditions or health or medical histories in a manner such that the individual users can be identified. Some users also do not want to publicize information related to hobbies and preferences. This has an influence on the amount of information publicized on the Internet that can be used as big data.

The present invention has been made in view of the disadvantages as described above. It is an objective of the invention to provide an information processing system to publicize the individual-related information so that the individual cannot be identified.

In order to solve the above disadvantages, an information processing system according to one embodiment of the present invention includes: a storage unit that is connected to the outside via a network and that stores individual-related information; an information publication unit; and a person meta data generation unit. The storage unit is configured to add a flag to each piece of individual-related information to show whether or not the information is publicized so that the individual cannot be identified. The person meta data generation unit is configured to generate the person meta data based on the individual-related information showing by the flag that the information is publicized so that the individual cannot be identified. The information publication unit is configured so that the person meta data can be accessed and publicized via the network.

According to the present invention, the information processing system can be provided that publicizes the individual-related information so that the individual cannot be identified.

### BRIEF DESCRIPTION OF DRAWINGS

FIG. 1 is a schematic view illustrating the configuration of an SNS information processing system;

FIG. 2 illustrates one example of user information stored in the database 102;

FIG. 3 illustrates an example of the user information publicized by the information publicizing server 104;

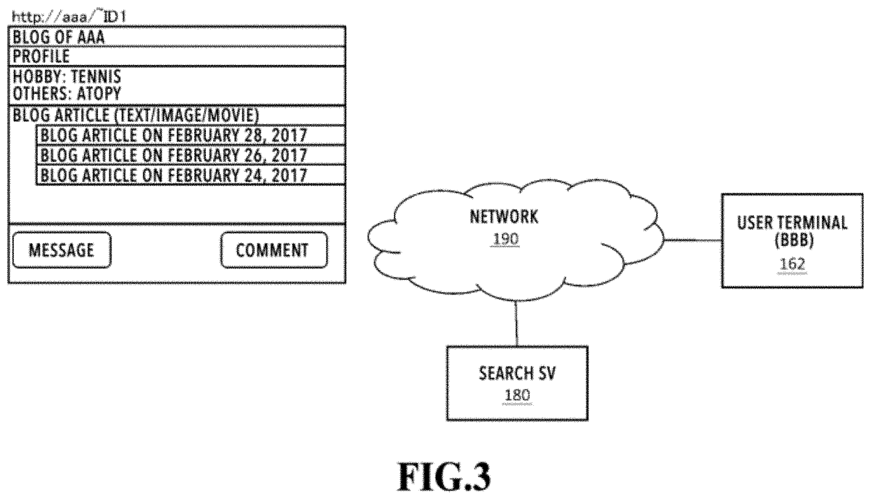

FIG. 4 is a schematic view illustrating the configuration of the SNS information processing system according to one embodiment of the invention of this application;

FIG. 5 illustrates one example of the user information stored in the database 102 of one embodiment of the invention of this application;

FIG. 6 illustrates one example of the user information stored in the database 102 of one embodiment of the invention of this application;

FIG. 7 illustrates an example of the user information publicized by the information publicizing server 104 of one embodiment of the invention of this application;

FIG. 8 is a schematic view illustrating the configuration of a hospital information processing system according to one embodiment of the invention of this application;

FIG. 9A illustrates one example of patient information stored in a patient information database 802 according to one embodiment of the invention of this application and illustrates an example of diagnosis record information;

FIG. 9B illustrates one example of the patient information stored in the patient information database 802 according to one embodiment of the invention of this application and illustrates prescription information;

FIG. 9C illustrates one example of the patient information stored in the patient information database 802 according to one embodiment of the invention of this application and illustrates the prescription information;

FIG. 9D illustrates one example of the patient information stored in the patient information database 802 according to one embodiment of the invention of this application and illustrates the prescription information;

FIG. 10A illustrates person meta data according to one embodiment of the invention of this application and illustrates the person meta data corresponding to FIG. 9A;

FIG. 10B illustrates the person meta data according to one embodiment of the invention of this application and illustrates the person meta data corresponding to FIG. 9B;

FIG. 10C illustrates the person meta data corresponding to one embodiment of the invention of this application and illustrates the person meta data corresponding to FIG. 9C;

FIG. 10D illustrates the person meta data corresponding to one embodiment of the invention of this application and illustrates the person meta data corresponding to FIG. 9D; and

FIG. 11 illustrates a processing flow in the information processing system of the present invention.

### DESCRIPTION OF EMBODIMENTS

Hereinafter, an embodiment of the present invention will be described in detail with reference to the drawings in which the same or similar reference numerals denote the same or similar components, and repeated description will be omitted. The embodiment described below is one example of the invention of this application. Thus, the invention of this application is not limited to the following embodiment and can be carried out in other embodiments without loss of generality.

The information processing system according to the embodiment of the present invention is an information processing system connected to the outside via a network. This information processing system includes a memory to store individual-related information, an information publicizing server (SV), and a person meta data generator. The memory stores each piece of individual-related information attached with a flag showing whether or not the information is publicized so that the individual cannot be identified. The person meta data generator generates the person meta data based on the individual-related information showing by the flag that the information is publicized so that the individual cannot be identified. The information publicizing server publicizes the individual-related information not shown by the flag so that the individual cannot be identified and the person meta data so that the former and the latter can be separately accessed via the network.

The person meta data includes the entirety or a part of back data of a certain person including, for example, the individual information of the person showing the nature of the person (e.g., the person's character, hobbies, blood type, height, weight, and medical history such as the one related to atopy, personal history, contact address). The person meta data can be used to represent the more-detailed nature of the person for example in addition to information generally used to identify the person (e.g., the person's individual number, name, and address). Data of information to identify a certain person (e.g., the individual number, name, and address) (hereinafter also may be referred to as main data) and the person meta data also may be configured separately and the former and the latter may be mutually associated. In another example, another configuration may be used in which the individual number is included in the main data and the person meta data includes the name and the address together with other information elements. In this case however, the information identifying the person such as a name and an address for example is generally not-publicized person meta data.

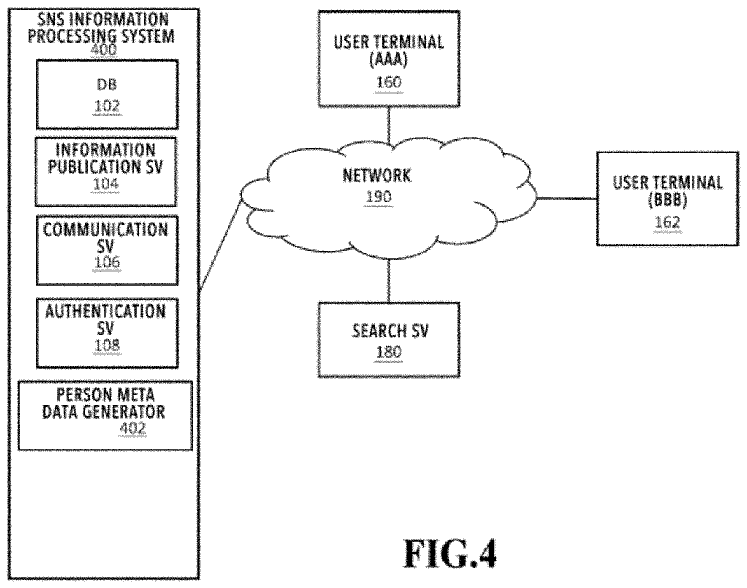

FIG. 4 is a schematic view illustrating the configuration of the SNS information processing system according to one embodiment of the invention of this application. An SNS information processing system 400 can be connected to the user terminals 160 and 162 as well as the search server 180 via the network 190. The SNS information processing system 400 shown in FIG. 4 can be configured by one or more computers including a processor such as a CPU; a semiconductor memory or a magnetic or optical memory; a wired or wireless communication device; an input apparatus such as a keyboard, an input pad, a mouse pointer, or a microphone; and an output apparatus such as a display, a printer, or a speaker.

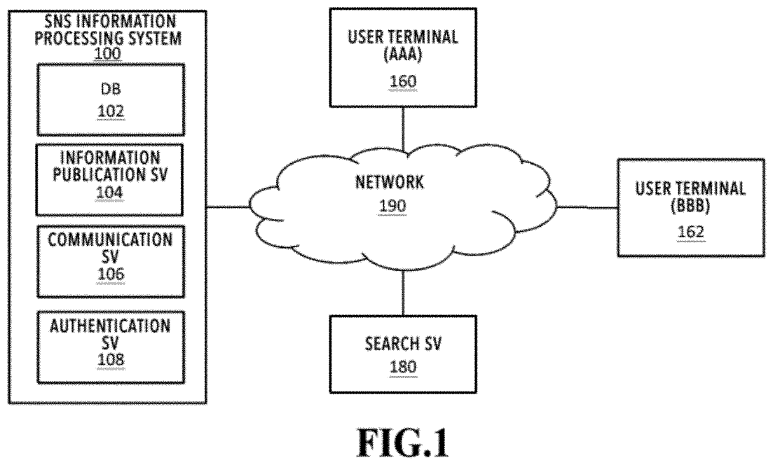

As in FIG. 1, the SNS information processing system 400 includes: the database 102; the information publicizing server 104; the communication server 106; and the authentication server 108. As shown in FIG. 4, the SNS information processing system 400 of this embodiment further includes the person meta data generator 402.

The database 102 is data to store the individual-related information of the respective users of the SNS system. The database 102 is retained in a memory.

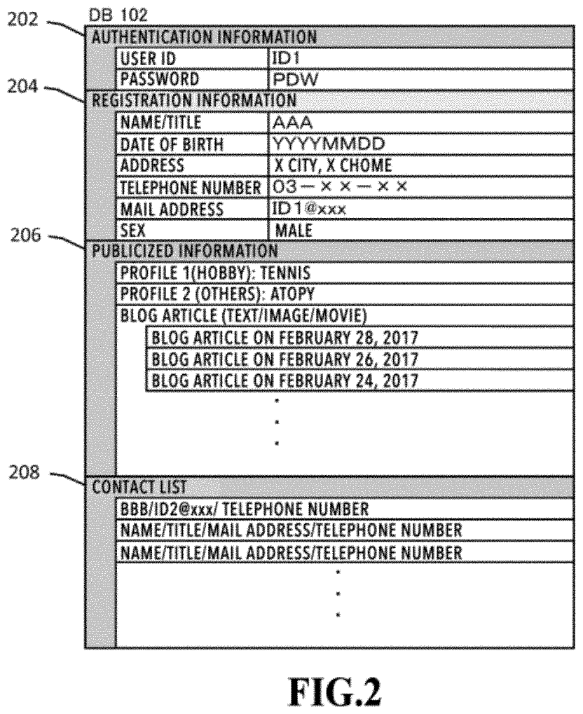

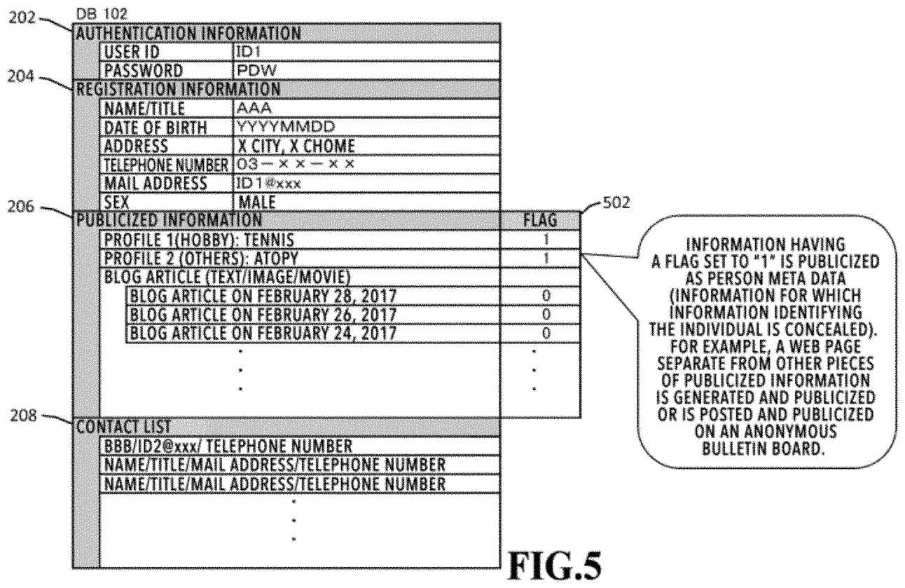

FIG. 5 illustrates one example of the user information stored in the database 102 according to this embodiment. As in FIG. 2, the database 102 includes: an authentication information 202 (user ID, password), a registration information 204 (name/title, date of birth, address, telephone number, mail address, sex), and the publicized information 206 (e.g., profiles 1, 2, blog article). The database 102 can include the contact list 208 (e.g., names/titles, mail addresses, telephone numbers of other users). As shown in FIG. 5, the database 102 according to this embodiment includes a flag 502.

The flag 502 shows whether or not each individual-related information stored in the database 102 is publicized so that the individual cannot be identified. For example, information for which the flag is set to “1” (e.g., profiles 1 and 2) shows that the person meta data (i.e., information identifying the individual) is publicized as concealed information as described later. Information for which the flag is set to “0” (e.g., a blog article) is publicized in a conventional manner. The information for which the flag is set to “1” (e.g., profiles 1 and 2) is publicized separately from the information for which the flag is set to “0”. For example, the former and the latter are publicized in different designs/formats and on different servers or addresses, respectively. When one of the information for which the flag is set to “1” and the information for which the flag is set to “0” is accessed, the former and the latter are not provided while being associated to each other.

In the above example, the flag “1” is associated with the publicized profile but also may be associated with another information category. For example, the respective flag values may be associated with a publicized information type so that “1” is associated with publicized “medical data”, “2” is associated with publicized “hobby”, “3” is associated with publicized “foot preference” for example. This association allows the publicized information to be searched in an organized manner.

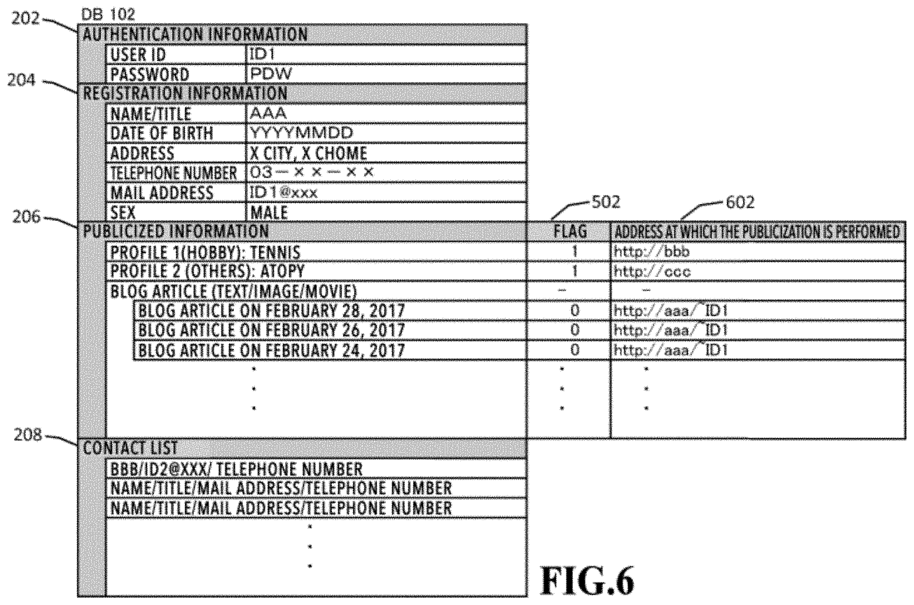

In the above description, an example was described in which a flag was used in order to identify whether the information is publicized or not and to identify the information category. Another example also may be used in which a flag can be used to identify whether the information is publicized or not and to identify those who is permitted to access the information (publication target). For example, as shown in FIG. 6, Table 1 shows that flags are set to be associated with the registration information stored in the database 102. Table 1 shows that the flag values “0” to “5” are associated in advance with whether the information is publicized or not and those who is permitted to access the information as shown in Table 2 (publication target), respectively.

TABLE 1

| Registration information | | Flag |
|---|---|---|
| Name | Yamada Taro | 0 |
| Address | Kanagawa-Ken | 0 |
| Mail | a@bbb.jp | 1, 2, 5 |
| Profile 1 | Tennis | 1, 2, 3, 4, 5 |
| Profile 2 | Atopy | 3, 4 |
| Food preference (like) | Eel | 1, 2, 5 |
| Food preference | Parsley | 1, 2, 5 |
| (dislike) | | |
| Favorite color | Yellow | 1, 2, 5 |

TABLE 2

| | Whether the information is publicized or |
|---|---|
| Flag | not/publication target |
| 0 | Not to be publicized |
| 1 | To publicize the person meta data to medical |
| | personnels |
| 2 | To publicize the person meta data to food |
| | manufacturer personnels |
| 3 | To publicize the person meta data to publishing |
| | personnels |
| 4 | To publicize the person meta data to health |
| | food manufacturer personnels |
| 5 | To publicize the person meta data to sporting |
| | goods manufacturer personnels |

In the example shown in Table 1, the person meta data is prepared and publicized so that the mail, food preference (like), and favorite color can be accessed by the medical personnels, food manufacturer personnels, and sporting goods manufacturer personnels and cannot be accessed by the publishing personnels, health food manufacturer personnels, and others. Alternatively, instead of the preparation of the person meta data, when the registration information shown in Table 1 is publicized by the information publicizing server 104, it is determined to which flag a person accessing the information corresponds (or it is determined to which registration information the access authorization is allocated) to provide, based on the determined flag, an access to the registration information. For example, such information is provided that is used to allow those who trying to access the information to determine whether they have a right to access from the user terminals 160 and 162 to the search server 180 or the information publicizing server 104 (e.g., information showing the type of occupation or the industry type for example). The search server 180 provides, to the information publicizing server 104, information used to determine the access right from the user terminal. When it is determined based on the provided information that a person trying the access is a medical personnel and corresponds to the flag “1”, then the information publicizing server 104 provides information registered as the one showing the mail, the profile 1, the favorite color (like), the color preference (dislike), and the color preference. Information registered as the one showing the name, the address, and the profile 2 may be provided while being masked. In this example, the medical personnels and the food manufacturer personnels for example were shown. However, the flag is not always limited to the occupation type and the industry type for example and also can represent the authorization or type of a person trying to access the data.

Alternatively, publicized information can be identified so that an individual cannot be identified without the use of a flag. For example, a specific data item of a database record is predetermined as the one to be publicized so that an individual cannot be identified. In this case, the person meta data generator generates the person meta data including the contents of the data item that is publicized by the information publicizing server. For example, a method may be used to set in advance, on a program, that the profile 1 and the profile 2 of FIG. 5 are publicized. Alternatively, all items other than the name, the address, the telephone number, and the mail address can be determined as publicized data. In this case, methods may be used such as a method to generate and publicize the person meta data of only the publicized information in a manner described below and a method to publicize the original data as an item while concealing the contents such as the name, the address, the telephone number, and the mail address.

In the example shown in Table 1, it was described that the flag 502 is set, for each piece of individual-related information stored in the database 102, whether or not the individual cannot be identified. However, as described above, the main data including a data item for identifying a certain person having an individual number, a name, and an address for example is configured separately from the person meta data that can be used to represent the more detailed nature of the person to associate the former and the latter. For example, when a certain person has an individual number 0001, an identifier A001 is given to the main data of the person and an identifier B001 is given to the person meta data to store them in the database 102 so that the information publicizing server 104 publicizes the person meta data having B001 only. In this case, only the contents of the person meta data having B001 are publicized and the individual number 0001 included in the main data having A001 may be concealed.

Any method may be used so long as the method can store the main data and the person meta data separately and can associate the former and the latter. For example, a method may be used to separately store the main data and the person meta data in a tabular format to prepare another tabular format data that associates the main data and the person meta data having a tabular format as information of the same person. Instead of another tabular format data, another method may be used to connect and store an identifier A001 and an identifier B001 so that the series of identifiers can be used to identify the information of a certain person. The tabular format data and the series of identifiers function as a map showing a position where the data is stored for example.

As described above, the main data is associated with the person meta data by another tabular format data or the series of identifiers. This association allows, even in the case of data leakage, the leakage can occur only in the main data, the person meta data, or another tabular format data, or the series of identifiers. Thus, the leaked data has a little significance and is hard to be utilized for unauthorized users, thus causing a reduced damage and providing a safe security. The person meta data may be further divided to provide divided pieces of the person meta data to correspond to an individual number, the name(s), or the address(s) (or a combination of the name and the address). This is safer because the respective pieces of the individual information are divided to pieces of the person meta data. Alternatively, another tabular format data or the series of identifiers may include a flag that shows whether or not the person meta data is publicized. By doing this, whether the person meta data is publicized or concealed can be specified not by the person meta data but by another tabular format data or the series of identifiers.

FIG. 6 illustrates one example of the user information stored in the database 102 of one embodiment of the invention of this application. FIG. 6 illustrates an example in which information having a flag set to “0” is publicized by an address obtained by combining a server address aaa with ID1 that is the user ID of the user AAA and information having a flag set to “1” (e.g., profiles 1 and 2) is publicized by separate addresses, respectively. The information publicizing server 104 or another element is configured so as to determine a server and an address to publicize the information having the flag “1”. The information publicizing server 104 or another element may refer to a list of a plurality of servers and addresses publicizing the person meta data (a list stored in the memory) to randomly determine, from the list, a server and an address publicizing each piece of person meta data. Such a list may be stored so as to include the association of themes or classifications regarding the details of the person meta data publicized by the respective servers. In this case, the information publicizing server 104 or another element may be configured, regarding the contents of the person meta data to be publicized, to perform an analysis (e.g., morphological analysis) and a decision (e.g., statistical decision and/or learning model-based decision) to determine the theme or classification to select a server and an address matching the theme or classification from the list. In order to select a server and an address based on the contents of the person meta data to be publicized, the person meta data having the same or similar theme or classification of a plurality of users (or a plurality of individuals) is collected and publicized with regard to the same server and address.

The person meta data generator 402 can be implemented by a processor executing a program stored in the memory. The person meta data generator 402 generates the person meta data based on the individual-related information to be publicized by a flag so that the individual cannot be identified.

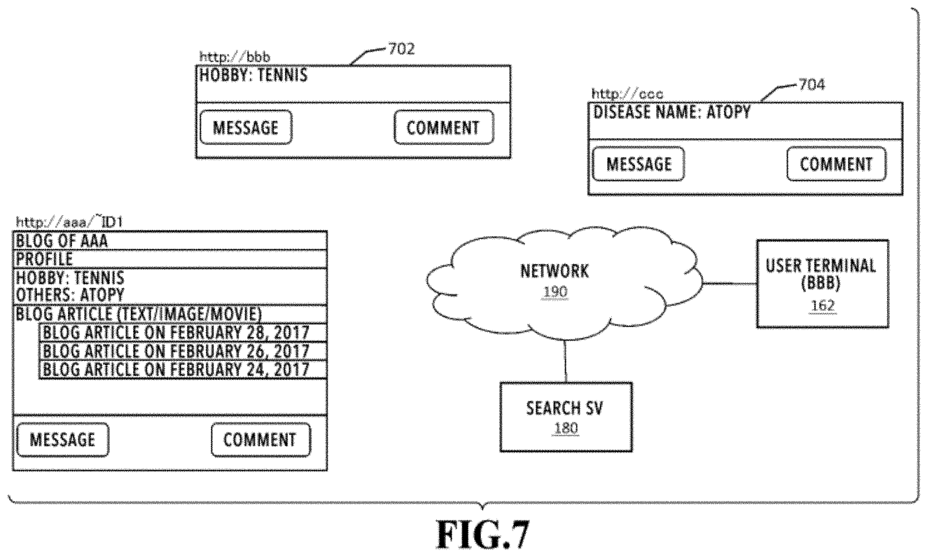

FIG. 7 illustrates an example of information publicized by the information publicizing server 104 of one embodiment of the invention of this application. Among the user information shown in FIG. 6, the person meta data 702 and 704 generated from information having the flag set to “1” is publicized together with different servers and addresses (“bbb” and “ccc”), respectively. Information having the flag set to “0” on the other hand is publicized as the blog of AAA having the address “aaa/ to ID1”. For example, the user BBB registered in the contact list of the user AAA can have an access from the user terminal 162 to the person meta data 702 and 704 as well as the information of the blog. The user BBB can recognize that the blog shows the information for the user AAA but cannot recognize for whom the person meta data 702 and 704 is described.

As shown in FIG. 7, the information publicizing server 104 publicizes the person meta data 702 and 704 together with a region (button) that can be activated to present a user interface UI to prepare a message or a comment for the person meta data on the user terminals 160 and 162 (laptop PC, tablet PC, mobile terminal, mobile phone, smart phone) of the user having accessed the person meta data 702 and 704. The user terminal displays the UI to prepare a message or a comment so that the address information of the user (individual) having provided the information from which the person meta data is originated is not identified or address information not belonging to the individual (e.g., an address exclusively used to send a message) is identified. For example, a message including an address at which the person meta data is publicized is received by the communication server 106 of the SNS information processing system 400 and a comment is stored in the server (bbb or ccc) publicizing the person meta data as log information associated with the person meta data. The communication server 106 may use the address information of the individual registered in the database 102 to notify an individual who cannot be identified through the person meta data of the existence of the message or comment regarding the person meta data.

In the above embodiment, a configuration was described in which the SNS information processing system 100 is connected to the search server 180 at the outside via a network. Alternatively, the SNS information processing system 100 may include the search server 180.

Alternatively, the above-described flag may be substituted with a configuration in which a specific region stored in the database 102 is specified in advance and the person meta data is generated.

Alternatively, the above region that can be activated to present the user interface may be substituted with an address to which a message or a comment is sent (address information not belonging to the individual (e.g., an address exclusively used to send a message)).

As described above, this embodiment can provide the SNS information processing system that publicizes the individual-related information so that the individual cannot be identified. The individual-related information publicized so that the individual cannot be identified promotes, for example, the publicization of physical feature information such as the one for a physical condition or health or medical history-related information. For example, if a need arises to know information regarding a medical history or a special hobby, a conventional case requires the notification of the existence of the medical history or special hobby, thus causing a risk where such a medical history or special hobby may be known to others for example. On the other hand, the SNS information processing system of this embodiment reduces such a risk by publicizing the person meta data. Specifically, the publicization of information is promoted, thus providing the improved use of big data. Furthermore, a message or a comment can be provided for the publicized person meta data, thus increasing the feedback for the information regarding the medical history or special hobby.

In the above embodiment, an example of the SNS information processing system was described. The invention of this application is not limited to the SNS information processing system. The invention of this application also can be used in many other embodiments so long as the person meta data can be separately retained. For example, the invention of this application applied to the individual-related information owned by companies, institutions, and public offices may provide the use of the person meta data as big data in such a manner that only the person meta data is publicized or shared or exchanged with others. For example, the invention of this application can be carried out as a medical or nursing-related information system. The following section will describe an example in which the invention of this application is carried out as a hospital-related information processing system (hereinafter will be called a hospital information processing system).

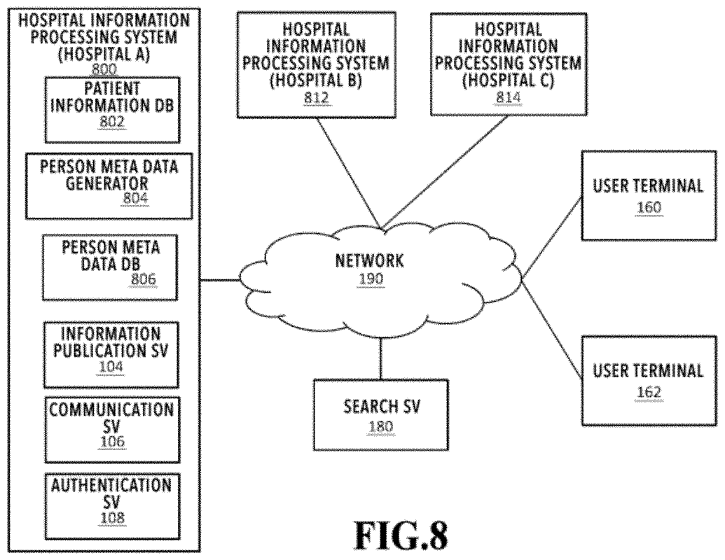

FIG. 8 is a schematic view illustrating the configuration of the hospital information processing system of one embodiment of the invention of this application.

The hospital information processing system 800 can be connected to the user terminals 160 and 162 as well as the search server 180 via the network 190. The hospital information processing system 800 can be configured by one or more computers including: a processor such as a CPU; a semiconductor memory or a magnetic or optical memory; a wired or wireless communication device; an input apparatus such as a keyboard, an input pad, a mouse pointer, or a microphone; and an output apparatus such as a display, a printer, or a speaker.

The hospital information processing system 800 includes: the information publicizing server 104; the communication server 106; and the authentication server 108. The hospital information processing system 800 includes: the patient information database 802; the person meta data generator 804; and the person meta data database 806.

The patient information database 802 is a database to store patient information. The patient information database 802 is retained in a memory.

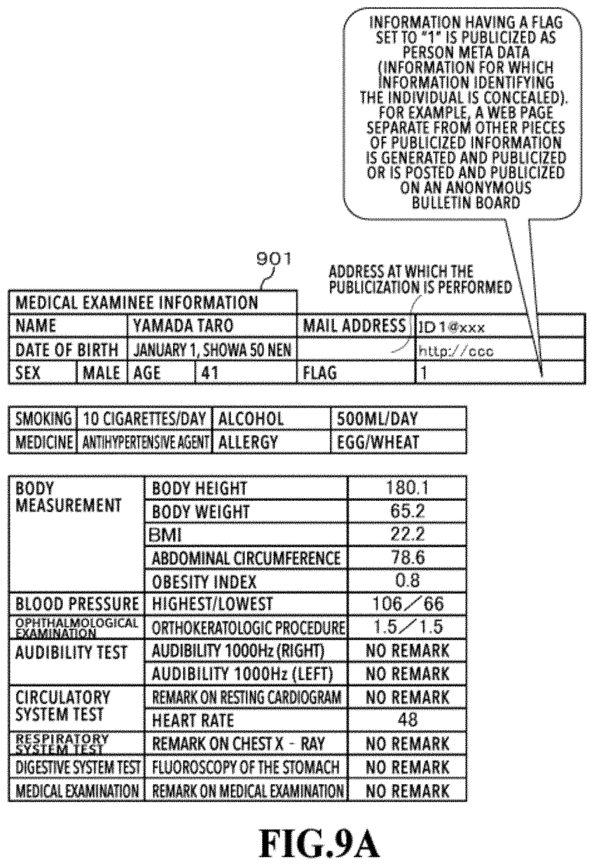

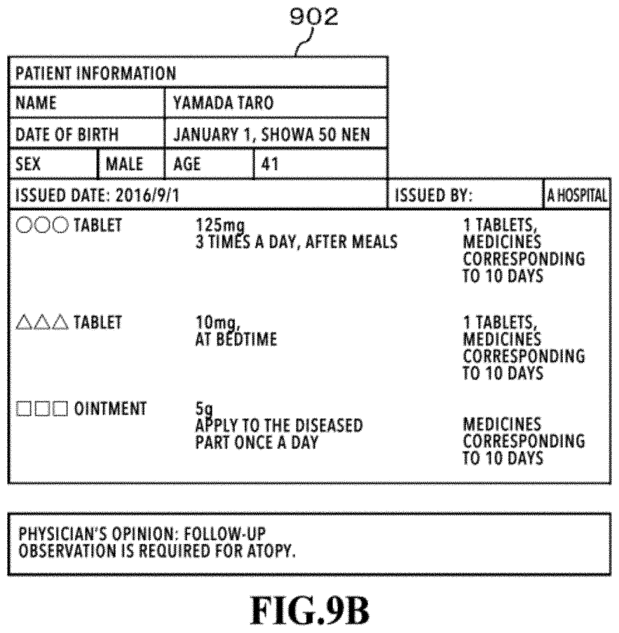

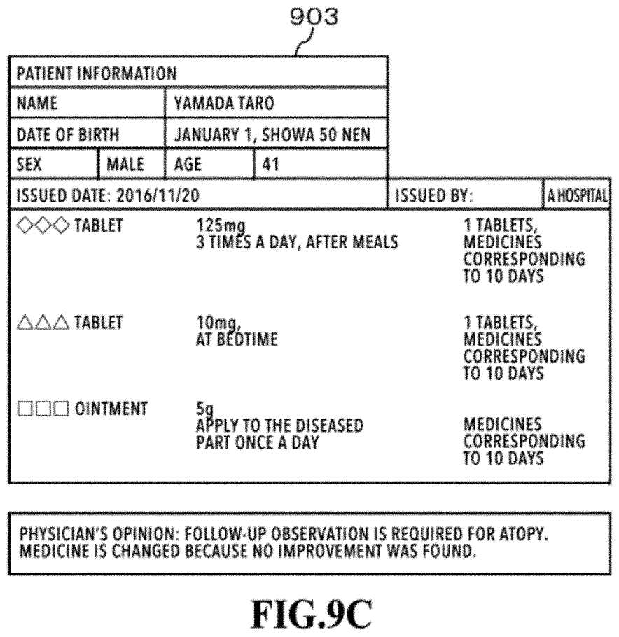

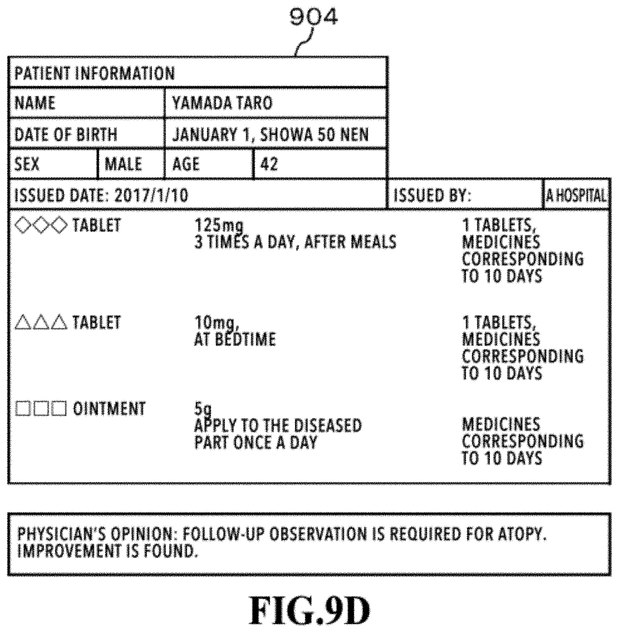

FIGS. 9A to 9D illustrate one example of patient information stored in the patient information database 802. FIG. 9A illustrates an example of the diagnosis record information 901. FIGS. 9B to 9D illustrate the prescription information 902 to 904 associated with the diagnosis record information 901. As shown in FIG. 9A, the diagnosis record information 901 is stored while being attached with a flag that shows whether or not the diagnosis record information and the prescription information regarding a patient as an individual are publicized so that the individual cannot be identified. The patient information having the flag set to “1” (the diagnosis record information 901, the prescription information 902 to 904) shows that the information is publicized as the person meta data (i.e., information for which information identifying the individual is concealed). The diagnosis record information 901 includes an address at which the patient information is publicized and a contact address (e.g., a mail address) of the patient to which the existence of a message or a comment regarding the publicized person meta data is notified.

The person meta data generator 804 can be implemented by a processor executing a program stored in the memory. The person meta data generator 804 generates the person meta data based on the individual-related information to be publicized by a flag so that the individual cannot be identified (the diagnosis record information 901 and the prescription information 902 to 904 of the patient). For example, the person meta data generator 804 can generate the person meta data by deleting, from the patient information, such information that identifies the individual (name, date of birth, mail address). The person meta data generator 804 also can generate the person meta data by substituting the information identifying the individual (name) with a character string of alphanumeric characters.

The person meta data database 806 stores the generated person meta data. The person meta data database 806 is retained in the memory.

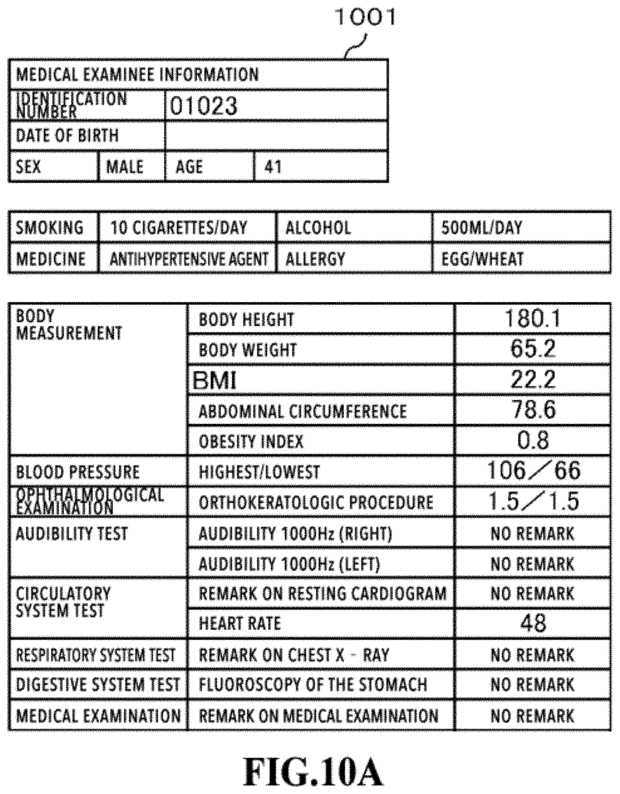

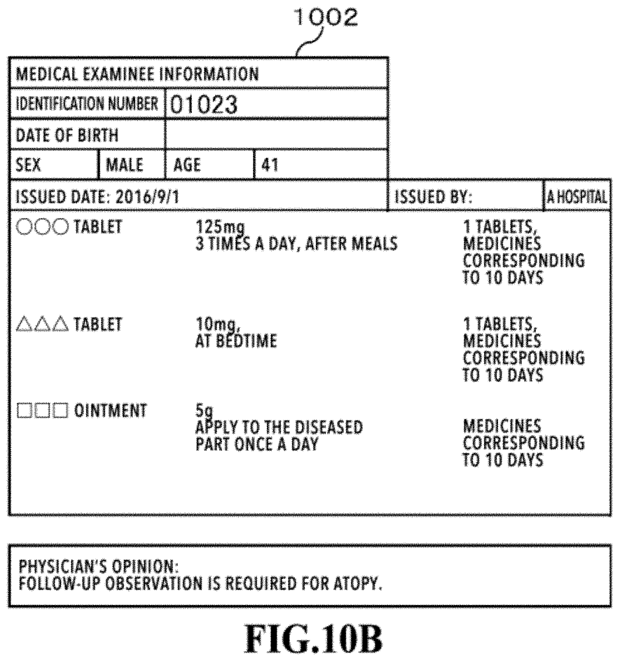

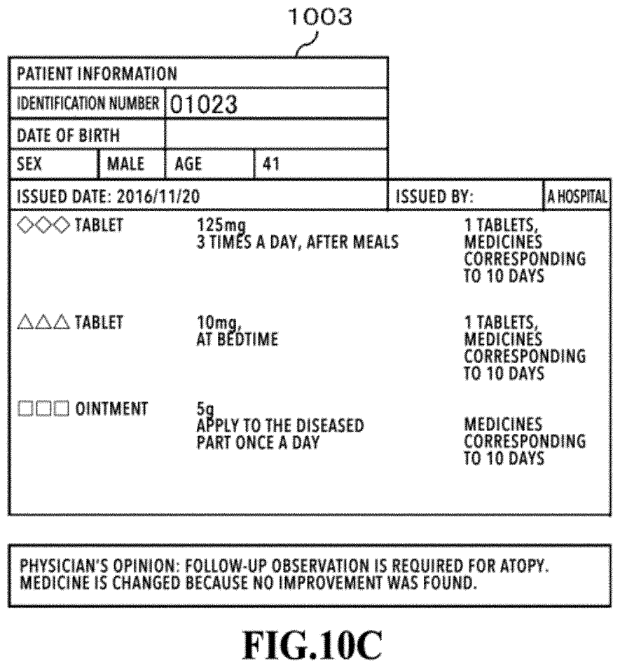

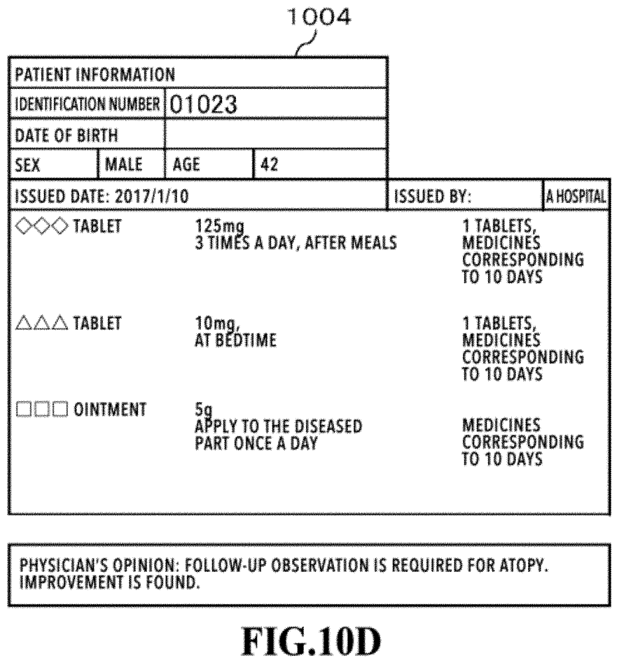

FIGS. 10A to 10D illustrate the person meta data according to one embodiment of the invention of this application. FIGS. 10A to 10D illustrate the person meta data 1001 corresponding to the diagnosis record information 901 of FIG. 9A and the prescription information 902 to 904 of FIGS. 9B to 9D, respectively. The person meta data 1001 of FIG. 10A deletes the date of birth of the diagnosis record information 901 and substitutes the name with a number to thereby delete the information identifying the individual (patient). The person meta data of FIGS. 10B to 10D is the person meta data corresponding to the prescription information 902 to 904 of FIGS. 9B to 9D, respectively. The information identifying the individual (patient) is similarly deleted from the person meta data of FIGS. 10B to 10D by deleting the date of birth of the prescription information of FIGS. 9B to 9D to substitute the name with a number. It is advantageous to use the person meta data including the details of the health status of the patient. For example, a more-appropriate understanding of a medicine actually taken by a patient may allow a pharmaceutical company to advantageously develop a new medicine.

As has been described with reference to FIG. 7, the information publicizing server 104 of the hospital information processing system 800 can publicize the person meta data 1001 to 1004. The person meta data 1001 to 1004 may be publicized together with a region (button) to present, on a user terminal of a user providing an access, a user interface to prepare a message or a comment regarding the person meta data. The pharmaceutical company can access the person meta data 1001 to 1004 that is a search result through the user terminal 162 via the search server 180 for example. Then, the user terminal 162 of the pharmaceutical company is displayed so that a message or a comment can be prepared without identifying the address information of the patient(individual) having provided the patient information from which the person meta data or while identifying address information not belonging to the patient (e.g., an address of the hospital information processing system 800 to exclusively send a message). The pharmaceutical company can use this user interface to prepare a message or a comment regarding the meta data. For example, a message including an address (http://ccc) at which the person meta data is publicized is received by the communication server 106 of the hospital information processing system 800. A comment is stored in a server (ccc) at which the person meta data is publicized as log information associated with the person meta data. The communication server 106 of the hospital information processing system may use patient address information (ID1@xxx) registered in the patient information database 802 to notify an individual who cannot be identified through the person meta data of the existence of the message or comment regarding the person meta data. The patient can access the message or comment regarding the person meta data through the user terminal 160 for example to confirm the contents.

As described above, the information publicizing server 104 or another element may determine the theme or classification of the contents of the person meta data 1001 to 1004 to be publicized through an analysis (e.g., morphological analysis) and a decision (a hard decision or a soft decision) to select matching server and address from the list. In this case, the same server and address collects and publicizes the person meta data of the same or similar theme or classification of a plurality of users (a plurality of individuals). For example, atopy-related person meta data is collected by the server (ccc). As described above, the server and the address selected based on the theme or classification provides the collection of the person meta data of the same theme or classification at the same server and address. However, the same server may include a plurality of different regions in which the person meta data having different themes or classifications may be collected.

As shown in FIG. 8, the search server 180 can access the person meta data publicized by a plurality of hospital information processing systems 800, 812, and 814 to search these pieces of person meta data in an inter-disciplinary manner. The search server 180 may be provided in at least one of the plurality of hospital information processing systems 800, 812, and 814.

In the above description, an example was described in which the hospital information processing systems 800, 812, and 814 include the person meta data databases 806, respectively. However, the search server 180 at the outside also may include the person meta data database 806 and the person meta data generator 804 of each hospital information processing system may store the generated person meta data in the person meta data database 806 at the outside. In this case, the hospital information processing systems 800, 812, and 814 can additionally store contact address (mail address) so that a notification regarding the provided meta data can be received from the search server 180 at the outside. The search server 180 at the outside provides a search service in the person meta data database 806 to provide the person meta data as a search result. When a message or a comment regarding the person meta data is generated, the search server 180 at the outside can acquire a contact address stored together with the person meta data to notify the address to the hospital information processing system. For example, the notification can include an identification number included in the person meta data. The notification may include a message or a comment or may include a procedure to access the message or the comment. The communication server 106 of the hospital information processing system can acquire, from the patient information database 802, a patient contact address from which the person meta data is originated (e.g., a mail address) to notify the patient of the existence of a message or a comment regarding the person meta data. As described above, the person meta data generator 804 of each hospital information processing system can store the generated person meta data in the person meta data database 806 of the search server 180 at the outside. Each hospital information processing system may include the search server 180 and the person meta data generator 804 may store the person meta data in a different space in the search server. The search server 180 at the outside may store the person meta data database 806 so that the person meta data is attached with string-type additional information obtained by combining an identifier for identifying the theme or classification of a data item and an identifier for identifying whether or not the publication is permitted and for identifying a publication target. The search server 180 can use the additional information as a substitute of the above-described flag or as a combination therewith. For example, the registration information of Table 1 and the additional information corresponding to the flag can be configured in a string-like manner in the manner described below: A:Yamada Taro:0; B:Kanagawa-Ken:0; B:a@bbb.jp:125; C:Tennis:12345; D:atopy:34; E:eel:125; F:parsley:125; G:Yellow:125. The data can be stored in the person meta data database 806. The reference numerals A to G show an example of an identifier to identify a theme or a classification. The reference numeral A shows an identifier corresponding to the name. The reference numeral B shows an identifier corresponding to contact information. The reference numeral C shows an identifier corresponding to a hobby. The reference numeral D shows an identifier corresponding to a physical condition. The reference numerals E to G show an identifier corresponding to like/dislike. The additional information has a string “D:atopy:34” that shows that the third data item in the data record has a theme or classification corresponding to a physical condition, the data value is “atopy”, and the publicization to “publishing personnels” and “health food personnels” is permitted. In order to permit the publicization of the information for physical condition-related theme or classification only to medical personnels, the string in the additional information corresponding to the third data item in the data record may be set as “D:atopy:1”. Alternatively, instead of storing a data value in each string of the additional information, an address (pointer) in a recording medium may be stored in which the data value is stored. This approach allows, even when a place where the data is stored is known, a system having the person meta data database 806 in which the person meta data is stored (e.g., hospital information management systems 800, 812, and 814, the search server 180) to also refer to a string in the additional information to deny or limit the access to the person meta data. Thus, the person meta data can be publicized or concealed in a more free manner. Even when the string in additional information is attacked from the outside, more safety is secured because the contents is not a data value but an address (pointer) in the recording medium in which the data value is stored.

As described above, this embodiment can provide the hospital information processing system that publicizes patient (individual)-related information so that the patient cannot be identified. Thus, the use of big data may be considered because the patient information can be publicized so that the patient cannot be identified. Furthermore, the publicized person meta data can receive a message or a comment, thus providing an increased feedback to the patient.

In the embodiment of the hospital information processing system, an example was described in which a data item (main data) to identify a certain person such as a name and a data item (person meta data) that can be used to represent the person in a more detailed manner are included in one tabular format data. However, the main data and the person meta data may be separately configured and may be associated to each other. As described above, the main data and the person meta data for example may be separately stored in a tabular format. Then, tabular format data may be prepared and stored that has an association showing that these two pieces of tabular-format main data and tabular-format person meta data are information related to the same person. Alternatively, another tabular format data may be substituted with a combination of the identifier of the main data connected to the identifier of the person meta data. This combination is stored so that the series of identifiers identify the information of a certain person.

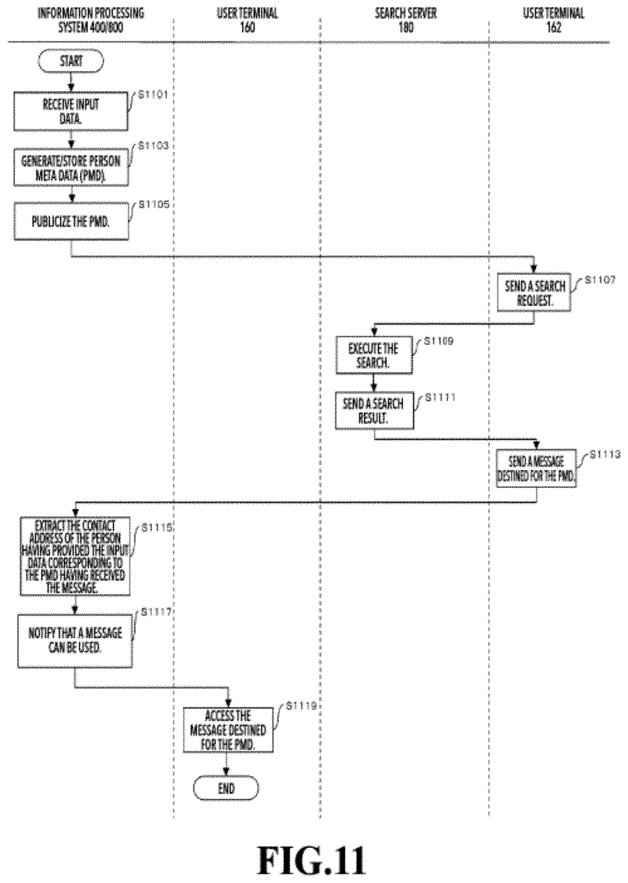

FIG. 11 illustrates a processing flow in the information processing system of the present invention. An information processing method shown in FIG. 11 can be carried out by the information processing system including the SNS information processing system 400 or the hospital information processing system 800 described above. The user terminal 160 is a terminal used by a user whose individual information is publicized as the person meta data for example. The user terminal 162 is a terminal used by a user to access meta data.

In S1101, the information processing system (communication server) receives and stores input data. For example, the SNS information processing system 400 communicates with the user terminal 160 operated by a user to receive the inputted profile data and the flag setting regarding the profile. The hospital information processing system 800 communicates with the user terminal 160 operated by a physician to receive inputted patient information (the diagnosis record information or the prescription information) and also receives the flag setting as required.

In S1103, based on the input data, the information processing system (the person meta data generator) generates person meta data. In S1105, the information processing system (information publicizing server) publicizes the generated person meta data.

In S1107, the user terminal sends a search request to a search server. In S1109, the search server executes the requested search. In S1111, the search server returns a search result to the user terminal. In S1113, the user terminal accesses the person meta data to generate and send a message or a comment via a user interface presented on the terminal.

In S1115, the information processing system (communication server or another element) extracts an address of a person who has provided input data corresponding to the person meta data having received the message or comment. The extraction of the address of the person having provided the input data can be performed in response to the reception by the information processing system (communication server or another element) of the message sent to the address of the information processing system or the detection of a record of the comment as a log in the server publicizing the person meta data.

In S1117, the information processing system (communication server) notifies that a message or a comment can be used.

In S1119, the user terminal accesses the message or the comment destined for the person meta data.

The information processing system (information publicizing server) may be configured, in response to the receipt of the request to access the publicized person meta data, to execute the authentication of a user associated with the request. The user authentication can be an authentication using an authorization child including a password authentication or biometrics authentication for example. The authorization child can be associated with information showing the occupation or industry type for example in advance. Similarly, the search server can authenticate a user associated with the search request prior to the execution of the requested search.

As described above, the invention of this application also can be carried out as an information processing method to publicize individual-related information so that the individual cannot be identified. the invention of this application also can be carried out as a computer program to allow a computer to execute this information processing method.

### REFERENCE SIGNS LIST

100, 400 SNS information processing system
102 Database
104 Information publicizing server
106 Communication server
108 Authentication server
160, 162 User terminal
180 Search server
190 Network
202 Authentication information
204 Registration information
206 Publicized information
208 Contact list
402 Person meta data generator
502 Flag
602 Address at which the publicization is performed
702, 704 Person meta data
800, 812, 814 Hospital information processing system
802 Patient information database
804 Person meta data generator
806 Person meta data database
901 Diagnosis record information
902, 903, 904 Prescription information
1001, 1002, 1003, 1004 Person meta data

1. An information processing system connected to the outside via a network, comprising: a storage unit for storing individual-related information; an information publication unit; and a person meta data generation unit, wherein:
the storage unit is configured to store each piece of the individual-related information together with a flag showing whether or not the information is publicized so that the individual cannot be identified,
the person meta data generation unit is configured to generate the person meta data based on the individual-related information shown by the flag that the information is publicized so that the individual cannot be identified, and
the information publication unit is configured to publicize the person meta data so that the person meta data can be accessed via the network.

2. The information processing system according to claim 1, wherein: the information publication unit is configured to publicize the individual-related information shown by the flag to be publicized so that the individual cannot be identified and the person meta data so that the former and the latter can be accessed separately via the network.

3. The information processing system according to claim 1, wherein: the information publication unit is configured to publicize the person meta data having the same or similar contents regarding a plurality of pieces of individual-related information collectively or in association with each other.

4. The information processing system according to claim 1, wherein: the information publication unit is configured to publicize the person meta data by providing the person meta data regarding a plurality of pieces of individual-related information to a storage apparatus included in a search system at the outside.

5. The information processing system according to claim 1, wherein:
the information processing system includes a communication unit,
the storage unit is configured to store address information of the individual,
the information publication unit is configured to publicize the person meta data together with a region that can be activated to present a user interface on a user terminal having accessed the person meta data to prepare a message or a comment regarding the person meta data,
the message or the comment is prepared while the address information of the individual not being identified or while address information not belonging to the individual being identified, and
the communication unit is configured to use the address information of the individual to notify the existence of the message or the comment regarding the person meta data to the individual who cannot be identified based on the person meta data.

6. The information processing system according to claim 1, wherein: the information processing system is an SNS information processing system.

7. The information processing system according to claim 1, wherein:
the information processing system is one or more medical, health, or welfare information processing systems, and
the individual-related information includes at least one of the diagnosis record information and the prescription information of patient.

8. The information processing system according to claim 1, wherein: the flag shows whether or not the information is publicized so that the individual cannot be identified and shows the type of information to be publicized.

9. The information processing system according to claim 1, wherein: the flag shows whether or not the information is publicized so that the individual cannot be identified and shows a publication target.

10. The information processing system according to claim 1, wherein: the person meta data includes, with regard to each piece of the individual-related information, string-type additional information obtained by combining an identifier for identifying the theme or classification of the individual-related information and an identifier for identifying whether or not the publication of the individual-related information is permitted and for identifying a publication target of the individual-related information.

11. An information processing method performed by an information processing system including a storage unit that stores each piece of individual-related information connected to the outside via a network together with a flag showing whether the information is publicized so that the individual cannot be identified, the method comprising:
generating the person meta data based on the individual-related information shown by the flag to be publicized so that the individual cannot be identified; and
publicizing the person meta data so that the person meta data can be accessed via the network.

12. A non-transitory computer readable storage medium storing a computer program for causing a computer including a processor and a memory that is connected to the outside via a network to perform operations comprising:
storing each piece of individual-related information together with a flag showing whether the information is publicized so that individual cannot be identified;
generating the person meta data based on the individual-related information shown by the flag to be publicized so that the individual cannot be identified; and
publicizing the person meta data so that the person meta data can be accessed via the network.

13. An information processing system connected to the outside via a network, comprising:
a storage unit for storing individual-related information;
an information publication unit; and
a person meta data generation unit,
wherein:
the person meta data generation unit is configured to generate person meta data based on the individual-related information to be publicized so that the individual cannot be identified, and
the information publication unit is configured to publicize the person meta data so that the person meta data can be accessed via the network.

14. The information processing system according to claim 13, wherein:
the information processing system includes a communication unit,
the storage unit is configured to store the address information of the individual;
the information publication unit is configured to publicize the person meta data together with an information region to present a user interface on a user terminal having accessed the person meta data to prepare a message or a comment regarding the person meta data,
the message or the comment is prepared while the address information of the individual not being identified or while address information not belonging to the individual being identified, and
the communication unit is configured to use the address information of the individual to notify the existence of the message or the comment regarding the person meta data to the individual who cannot be identified based on the person meta data.

15. The information processing system according to claim 13, wherein: the individual-related information to be publicized so that the individual cannot be identified is a specific data item of a database record regarding the individual-related information stored in the storage unit.
